(19) United States
(12) Patent Application Publication
(10) Pub. No.: US 2023/0363653 A1
(43) Pub. Date: Nov. 16, 2023

(54) WEARABLE VITAL SIGNS MONITORING DEVICE AND METHOD

(71) Applicant: HINLAB, Neuilly-Sur-Seine (FR)

(72) Inventors: Denys-Michel DE BEGON DE LAROUZIERE DE MONTLOSIER, Paris (FR); Aymeric PEIRS, Paris (FR)

(73) Assignee: HINLAB, Neuilly-Sur-Seine (FR)

(21) Appl. No.: 18/248,292

(22) PCT Filed: Oct. 8, 2021
(86) PCT No.: PCT/EP2021/077930

§ 371 Date: Apr. 7, 2023

(30) Foreign Application Priority Data
Oct. 9, 2020 (EP) 20306192.4

(51) Int. Cl. A61B 5/0225; A61B 5/022; A61B 5/00

(52) U.S. Cl. CPC A61B 5/02255; A61B 5/02233; A61B 5/6824; A61B 2560/0223

(57) ABSTRACT

A wearable device for blood pressure monitoring, which includes an armband with an inner surface and an outer surface, an inflatable bladder within the armband, a pumping element for inflating and deflating the inflatable bladder, an optical sensor and a pressure sensor. The optical sensor is at least partially exposed at the inner surface of the armband.

### FIELD OF INVENTION

The present invention pertains to the field of devices and methods for physiological parameter estimation. In particular, the invention relates to a wearable device for vital signs monitoring, such as blood pressure monitoring.

### BACKGROUND OF INVENTION

A non-invasive and accurate blood pressure estimation of a subject is needed in hospital and home settings. In some cases, a continuous or regular (e.g. hourly) blood pressure estimation is desired both in a hospital setting and in a home setting.

Traditional sphygmomanometers comprise an inflatable cuff configured to be worn around an arm of a subject. The cuff is inflated, so as to occlude the brachial artery, and then deflated, until the blood flow in the brachial artery is restored. The blood pressure of the subject wearing the cuff may be estimated with an oscillometric technique or an auscultatory technique. The former estimates the systolic and diastolic pressures based on the amplitude of the pressure oscillations recorded within the cuff during deflation or inflation, the latter estimates the systolic and diastolic pressures based on the Korotkoff sounds recorded with a stethoscope during the cuff deflation.

Finger and wrist sphygmomanometer have also been developed. However, these monitors are less accurate than upper-arm sphygmomanometer and, for this reason, the American Heart Association recommends not using them (Muntner, Paul, et al. “Measurement of blood pressure in humans: a scientific statement from the American Heart Association.” Hypertension 73.5 (2019): e35-e66.). In fact, finger sphygmomanometers underestimate the blood pressure and are sensitive to background noise, whereas wrist blood pressure monitors are subjected to a lack of accuracy due to their tendency not to maintain the proper positioning. Moreover, the accuracy of blood pressure measurements at the wrist is highly affected by the position of the wrist relative to the heart.

Cuff-less blood pressure estimating are popular outside the clinical setting. These devices rely on the use of an optical sensor placed on the skin of a subject; the blood pressure of the subject is then estimated based on the signal collected from the optical sensor. Because of the inherent lack of accuracy of cuff-less devices, their use is not recommended in all those settings in which an accurate blood pressure estimation is needed, such as for instance in the clinical settings.

Cuff-based monitors configured to estimate the blood pressure at the arm level have high accuracy; however, they cannot provide regular blood pressure recording combined with optical sensor data. Such monitors cannot monitor patient health reliably and detect the need for blood pressure measurements. Moreover, conventional cuff-based monitors are not configured to measure relevant physiological parameters other than blood pressure.

### SUMMARY

The present invention aims to provide a device for non-invasive estimation of various physiological parameters of a subject that solves the problems of the prior art, namely, the device provides a continuous, or regular, and accurate blood pressure monitoring, while avoiding discomfort for the patient. More in particular, an object of the present invention is a wearable device capable of continuously or regularly and accurately measure a blood pressure of a wearer and signal when such measurements are required.

It is also an objective of the present invention to provide a blood pressure monitoring device capable of calculating an accurate blood pressure of a subject autonomously, i.e., without a need for the subject to perform manual operations.

The present invention relates to a wearable device for blood pressure measurement comprising:
an armband with an inner surface and an outer surface;
a bladder within the armband configured to be inflated and deflated;
pumping means for inflating and deflating said bladder, the pumping means comprising a pump, a pipe in fluid communication with the bladder and a valve;
at least one optical sensor within the armband, the optical sensor being at least partially exposed at the inner surface of the armband, so as to permit acquisition of an optic signal from an arm of the subject wearing the armband;
at least one pressure sensor configured to acquire a signal indicative of a pressure within the bladder; and
a processing unit configured to:
   receive the signal from the optical sensor and the signal from the pressure sensor;
   calculate a first blood pressure value of the wearer based on the signal from the optical sensor, and a second blood pressure value based on the signal from the pressure sensor; and
   calibrate the first blood pressure value on the basis of the second blood pressure value.

The first blood pressure value is calculated by the processing unit based on the signal from the optical sensor, which in turn depends on blood volume and, more precisely, on the amount of blood in the optical path of the sensor.

As blood volume and blood pressure are closely related, the calculation of the first blood pressure value is accurate and does not require complex mechanistic models (e.g.: making assumptions about a time delay between these two quantities). Moreover, there is no need for patient cables, chest belt, or electrodes.

In a preferred embodiment, the first blood pressure is calculated directly from the optical sensor.

This embodiment is particularly advantageous as it does not require several sensors at different locations on the subject's body (as it is the case for electroencephalographic electrodes), thereby avoiding any discomfort to the subject, and reducing the risk of errors in the blood pressure estimation due to poor synchronization of the sensors.

The first blood pressure value thus obtained is a relative pressure. Indeed, as aforementioned, optical technologies such as photoplethysmography do not provide an absolute measure of blood pressure. These technologies allow to obtain may only provide blood pressure trends, i.e., relative pressures.

Inversely, the second blood pressure value is an absolute pressure.

Therefore, the device of the invention allows to provide automatic calibrations of the first blood pressure value using the second blood pressure value, thereby providing a continuous or regular monitoring of the absolute blood pressure of the wearer over time.

By automatic, or autonomous, calibration it is meant a calibration step which does not require human intervention. Neither the wearer nor healthcare providers has to perform any manual operation because, in order to perform a calibration, the present device (i) does not require the sensors to be displaced from their original position and (ii) does not require to be manually calibrated against a standard.

Therefore, the device is particularly advantageous for providing a blood pressure monitoring of hospitalized and/or bedridden persons.

In one embodiment, the device further comprises an acoustic sensor.

The acoustic sensor allows to detect the presence of Korotkoff sounds during inflation of deflation of the bladder.

In one embodiment, the pumping means are within the armband.

Advantageously, this allows to obtain a device of compact dimensions, which does not require multiple components placed at different location on the wearer's body.

Moreover, having the pumping means integrated in the armband allows to reduce the dead volume of the fluid pipe.

In one embodiment, the device further comprises a power supply device.

Preferably, the power supply device comprises a built-in battery and a removable battery.

In one embodiment, the removable battery is further configured to supply power to the built-in battery.

In this case, the built-in battery is configured to supply power to the wearable device in absence of a removable battery, for instance during the replacement of the removable battery.

This advantageously ensures uninterrupted power supply to the device, i.e. even when the removable battery is removed, thereby guaranteeing continuity of the blood pressure estimation and/or other vital signs monitoring.

However, in some embodiments the power supply device may comprise only a built-in battery or a removable battery.

In one embodiment, the device according to the present invention further comprises a memory configured to store a list of events, the list of events comprising at least one time interval Δt; and the processing unit is configured to:
retrieve, from the memory, the list of events and monitor the occurrence of said events;
in response to the detection of at least one of said events, trigger the inflation and deflation of the bladder by means of the pumping means;
receive the signal from the pressure sensor during the inflation and/or deflation of the bladder;
calculate the second blood pressure value of the wearer based on the signal from the pressure sensor.

In one embodiment, the processing unit is further configured to
execute a first algorithm configured to calculate the first blood pressure value of the wearer based on the signal received from the optical sensor;
execute a second algorithm configured to calculate the second blood pressure value of the wearer based on the signal received from the pressure sensor;
estimate a set of parameters of the wearer based on the first and the second blood pressure values.

In this case, the processing unit may be configured to calibrate the first blood pressure value based on the estimated set of parameters of the wearer.

For instance, the estimated set of parameters of the wearer may comprise parameters representative of hemodynamic property, mechanical property or geometrical property related to the arterial blood flow, and/or algorithmic parameters (e.g., machine learning features), and/or histogram statistics.

In one embodiment, the device further comprises at least one third sensor distinct from the aforementioned optical and pressure sensor.

The third sensor is preferably selected from a group comprising: an ultrasound sensor; an accelerometer; a gyroscopic sensor; an electrical sensor; an optical sensor; a tomographic sensor; an acoustic sensor, a magnetometer; a humidity sensor; and the processing unit is further configured to receive a physiological signal from said third sensor.

For instance, the device may further comprise a temperature sensor and/or a heat flux sensor. In this embodiment, the processing unit may be further configured to receive a signal from the temperature sensor and/or a signal from the heat flux sensor and to execute a third algorithm, so as to estimate a core body temperature of the wearer based on said received signal.

The present invention also relates to a method for continuous or regular monitoring of the blood pressure of a subject, the method being implemented by a device according to any one of the embodiments described hereinabove and comprising the following steps:
a) receiving, as input, a signal from the optical sensor;
b) calculating the first blood pressure value of the wearer by means of a first algorithm based on the signal received from the optical sensor;
c) receiving, as input, a signal from a pressure sensor during deflation and/or inflation of the bladder;
d) calculating the second blood pressure value of the wearer by means of a second algorithm based on the signal received from the pressure sensor;
e) calibrating the first blood pressure value on the basis of the second blood pressure value.

Advantageously, the method according to the invention allows to provide the blood pressure of the wearer and to this end it does not require a displacement the optical sensor, or other components. Therefore, it can be used to provide the blood pressure of a subject that could not execute said movement, for instance a subject having reduced mobility, immobility or pain; a subject that is asleep, under anesthesia or in a coma; a subject that cannot understand the request of displacing the sensor, such as an infant, or a person with impaired cognitive abilities.

Moreover, the method allows to automatically calibrate the first blood pressure value thereby avoiding the use of external calibration standards, which is time consuming, has to be performed by a dedicated individual (e.g., a technician), and it is not implementable in hospital routines.

The aforementioned list of events may comprise for instance a time interval. In this case, the device may continuously provide blood pressure values of the wearer over time using the optical sensor signal, and periodically calibrate those blood pressure values over time, at a constant calibration frequency.

In one embodiment, the method further comprises the following steps:
retrieving a list of events stored in a memory, wherein in the list of events each event is associated to at least one predefined threshold, and monitoring the occurrence of said events;
triggering the inflation and deflation of the bladder if the signal from the sensor and/or the first blood pressure value is above or below the predefined threshold.

This embodiment advantageously allows to trigger the inflation and deflation of the bladder in presence of alert or abnormal values.

Alert values may relate to sharp changes in the first blood pressure value, lack of signal or unexpected signal from additional sensors (e.g., temperature sensor, accelerometer, etc.), a change in pulse strength (i.e., amplitude of the optical sensor signal), change in pulse frequency, and the like.

The list of events may comprise for instance some thresholds (e.g., maximum amplitude, maximum coefficient of variation, maximum difference or ratio between a current value and a previous value for a predetermined signal metrics) for the sensor signals and/or for the first blood pressure value.

In this case, the frequency of calibration of the first blood pressure value may be variable, depending on the occurrence of said events.

In one embodiment, the method according to the present invention further comprises a step of receiving, as input, a signal from a third sensor, said third sensor being distinct from the optical sensor and the pressure sensor, and during the first calculation step the first blood pressure value of the wearer is calculated by means of a first algorithm based on the signal received from the optical sensor and/or on the signal received from the third sensor.

This embodiment allows to provide a first blood pressure value based on the optical sensor signal as well as additional physiological signals.

In one embodiment, the steps of the methods defined hereabove are preceded by a step of retrieving a set of parameters of the wearer from a memory. In this embodiment, during the first calculation step, a first blood pressure value of the wearer is calculated based on the signal received from the optical sensor and calibrated based on the retrieved set of parameters.

This embodiment allows to retrieve the results of a previous calibration (i.e., the set of parameters), and to calculate the first blood pressure value therefrom, which is particularly advantageous to derive an accurate blood pressure value from the optical sensor signal in between two calibrations.

In one embodiment, the steps of calculating a first and/or a second blood pressure values are performed by means of a machine learning algorithm.

For instance, the first blood pressure value may be obtained via a step of decomposition of the optical sensor signal into features and a subsequent feature selection step.

In one embodiment, the steps a) and b) are periodically repeated at a first repetition frequency and the steps c) to g) are periodically repeated at a second repetition frequency, the second repetition frequency being inferior to the first repetition frequency.

The present invention also relates to a computer program product for continuous or regular monitoring the blood pressure of a subject, the computer program product comprising instructions which, when the program is executed by a computer, cause the computer to carry out the steps of the method as described hereabove.

The present invention also relates to a computer-readable storage medium comprising instructions which, when the program is executed by a computer, cause the computer to carry out the steps of the method as described hereabove.

### BRIEF DESCRIPTION OF THE DRAWINGS

FIG. 1 is a block diagram schematically showing a device 1 according to the present invention.

FIG. 2 represents the device 1 of the present invention according to a first embodiment in which the power supply device 2, the pump 110, the valve 112, and the pressure sensor 13 are buried in the armband 10, i.e. they are positioned between the inner surface 103 and the outer surface 104, and wherein the power supply device 2 and the opening 100 are spaced apart along the armband length so that, when the device 1 is wrapped around an arm, said power supply device 2 and the opening 15 are diametrically opposed with respect to the arm. In FIG. 1, the outer surface is not illustrated for intelligibility purpose, so as to show the elements buried in the armband 10.

More precisely, FIG. 2A, FIG. 2B and FIG. 2C respectively are:
a side view of the present device 1 according to said first embodiment;
a perspective view of the present device 1 according to said first embodiment; and
a perspective view of the present device 1 according to said first embodiment.

FIG. 3 represents the device 1 of the present invention according to a second embodiment in which the pumping means 11 are buried in the armband 10, whereas the power supply device 2, and the pressure sensor 13 are exposed at the outer surface 104, and wherein the opening 100 and the power supply device 2 are not spaced apart along the armband 10 length; i.e., they are on the same side of an arm when the device 1 is wrapped around said arm.

More precisely, FIG. 3A and FIG. 3B respectively are:
a first perspective view representing the device 1 of the present invention according to the second embodiment; and
a second perspective view representing the device 1 of the present invention according to the second embodiment.
a first perspective view representing the device 1 of the present invention according to the second embodiment; and
a second perspective view representing the device 1 of the present invention according to the second embodiment.

FIG. 4 is a flow chart showing the main steps a method compliant with the present disclosure, which may be executed by the device 1 of FIG. 1.

FIG. 5 is a flow chart schematically showing two consecutive implementations of the method of the invention, according to one particular embodiment.

FIG. 6 shows the first blood pressure value estimated by the processing unit 15 of the device 1, as a function of time.

### DETAILED DESCRIPTION

The following detailed description will be better understood when read in conjunction with the drawings. For the purpose of illustrating, the device is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

The present invention relates to a wearable device for measuring the vital signs of a wearer. In a preferred embodiment, the present invention relates to a wearable device configured to provide a continuous, or regular, and accurate blood pressure monitoring.

In the following detailed description, the terms wearer, subject, and patient have the same meaning.

More in particular, the present device is capable of: estimating a first blood pressure value based on the signal received from an optical sensor 12, detecting the occurrence of an event indicating that a calibration is required, triggering a cuff-based blood pressure estimation and calibrating the first blood pressure value based on the result of the cuff-based blood pressure estimation. Therefore, device allows to perform said calibration at an optimal frequency, thus providing a continuous, or regular, and accurate blood pressure monitoring, while avoiding discomfort for the wearer.

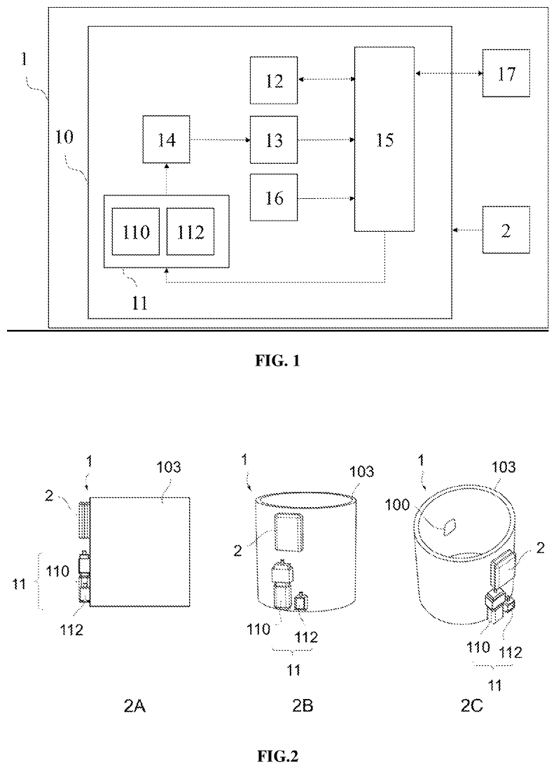

FIG. 1 is a block diagram illustrating the device 1 of the invention according to one particular embodiment.

The device 1 comprises several components: an armband 10 (FIG. 2), a bladder 14 located within the armband 10, pumping means 11 for inflating and deflating said bladder 14, at least one optical sensor 12, at least one pressure sensor 13, a processing unit 15.

As can be seen in FIG. 1, the sensors 12, 13 are communicatively connected to the input processing unit 15 of the device 1, for instance via a wired connection or wireless connection, so that the processing unit 15 can receive the measurements from the optical sensor 12 and the pressure sensor 13.

The processing unit 15 may communicate bi-directionally with the sensors 12, 13. For instance, the processing unit 15 may control one or more sensing operations, such as modulating the power or the light intensity of the optical sensor 12.

The device 1 of FIG. 1 also comprises a memory 16, a user interface 17 (via which information can be entered and/or retrieved by a user), and a power supply device 2.

Advantageously, the interface 17 permits to receive inputs or external commands (e.g., a request of inflating the bladder) from a user (e.g., a healthcare provider) and/or to output one or more vital signs of the wearer calculated by the processing unit 15.

The interface 17 may be for instance a hospital monitor, smartphone, a tablet computer, a portable computer, a laptop computer, or any other electronic devices capable of generating, receiving, or exchanging data, information or instructions over a wired or wireless network.

Moreover, in the example of FIG. 1, the pumping means 11, the pressure sensor 13, the processing unit 15, and the memory 16 are comprised in the armband 10.

However, in some embodiments each of these components may be external to the armband 10.

These components will be described in more details in the following.

### The Armband

The armband 10 of the device 1 is configured to be worn around an arm of the subject, preferably around the upper arm.

Advantageously, a wearable device 1 comprising an armband 10 is less sensitive to motion artifacts which may affect the sensor measurements, when compared to wearable devices worn around the wrist or a finger.

Moreover, the arm is characterized by elevated vascularization, and presence of main arteries such as the brachial artery.

Therefore, the blood pressure measured with the present device 1 is indicative of the blood pressure in the arm arteries, such as the brachial or humeral artery, which is close to the blood pressure in the aorta. This allows to provide a highly accurate and non-invasive blood pressure estimation.

The armband 10 is better shown in FIG. 2. More precisely, FIGS. 2A, 2B and 2C respectively are: a side view, a first perspective view, and a second perspective view of the wearable device 1.

The armband 10 has an inner surface 103 and an outer surface 104. The inner surface 103 is in contact with the skin of the wearer when the device 1 is worn.

In the example of FIG. 2A the pumping means 11 and the power supply device 2 are buried inside the armband 10, i.e., between the inner and the outer surfaces 103, 104.

In this example, the pumping means 11 comprise a pump 110 and a valve 112. These components are better shown in FIG. 2B.

As can be seen in FIG. 2C, the armband 10 also comprises an opening 100 configured to expose at least partially the optical sensor 12 at the inner surface 103 of the armband 10. This allows to acquire an optical signal from an arm of the subject wearing the armband 10.

Of note, the outer surface 104 of the armband 10 is not illustrated in FIGS. 2A to 2C, so as to show the pump 110, the valve 112 and the power supply device 2, which otherwise would be hidden by said outer surface 104. Moreover, the sensors 12, 13 are not represented.

The armband 10 has a height and a length transverse to the height.

The armband 10 length is long enough to cover the circumference of the upper arm of the subject.

In one embodiment, the armband 10 length is comprised between 20 cm and 42 cm, preferably between 20 cm and 36 cm or between 20 cm and 30 cm.

The armband 10 is high enough to receive at least an inflatable bladder 14 and sensors 12, 13, for instance provided in a sensor array.

According to one embodiment, the armband height is comprised between 5 cm and 16 cm, preferably between 9 cm and 14 cm.

In one particular embodiment, the armband length is equal to 26 cm and the armband height is equal to 12 cm.

In one embodiment, the armband 10 comprises a slot. In this embodiment, the inflatable bladder 14 may not extend throughout the length of the armband 10, and the slot may be in the armband 10 portion that is not occupied by the bladder 14.

For instance, a power supply device 2 may be removably disposed within said slot, whereas the sensors of the sensor array are embedded inside the armband 10.

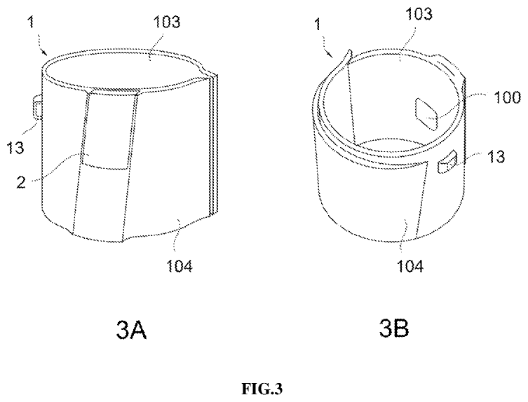

FIGS. 3A and 3B are perspective views of the armband 10 in which the outer surface 104 is also represented.

In one particular embodiment, one or more elements may be exposed at the outer surface 104 instead of being buried in the armband 10. For instance, the pressure sensor 13 may comprise a first portion buried inside the armband 10 and a second portion exposed at the outer surface 14. One example of this embodiment is represented in FIG. 3A and FIG. 3B.

The armband 10 may be configured to be wrapped around the subject's upper arm and to be fastened for instance with hook-and-loop attachment means.

In one embodiment, the armband 10 is configured to be fastened with a buckle or an adhesive. In one embodiment, the armband 10 is configured to be wrapped around the subject's upper arm and to be fastened with magnetic attachment means.

### The Bladder

As aforementioned, the device 1 according to the present invention comprises a bladder 14. Said bladder 14 is inflatable to a pressure that is high enough to uniformly restrict the blood flow in the subject's arm around which the armband 10 is worn.

In other words, the bladder 14 may be inflatable up to a maximum inflation pressure which is superior to the pressure needed to occlude the brachial artery.

Said maximum inflation pressure may be equal to 250 mm Hg, preferably 260 mm Hg, 2270 mm Hg or 280 mm Hg. In some cases, it may be desirable to provide a bladder 14 inflatable up to a pressure of 300 mm Hg.

According to one embodiment, the inflatable bladder 14 does not extend throughout the length of the armband 10. For instance, the inflatable bladder 14 may extend for a length between 20 and 42 cm preferably between 22 cm and 36 cm., so as to cover for instance 80% of the wearer's arm.

Alternatively, the bladder 14 may extend throughout the length of the armband 10.

The bladder 14 may be made of resin, rubber, polyester, synthetic fiber, or a combination thereof. Preferably, the bladder 14 is made of plastic fibers, TPU, PVC, or a combination thereof.

In some embodiments, the bladder may be made of coated fabrics such as Nylon™

### The Pumping Means

The present wearable device 1 also comprises pumping means 11 for inflating and deflating the bladder 14.

Said pumping means 11 may comprise a pump 110, a pipe in fluid communication with the bladder 14 and a valve 112.

For example, said pump 110 may be a rolling air pump, a piezo electric pump, a resonant gas pump, a diaphragm/membrane gas pump 110, and the like.

By valve 112 it is meant any component configured to control the passage of fluid through the pipe of the pumping means 11, for instance by opening, closing, or partially obstructing the pipe.

The valve 112 (or a valve assembly) may be physically separated from the pumping means 11.

Alternatively, the valve 112 may be integrated in the pumping means 11. For instance, the pump 110 may comprise one or more blowers, which allows to discharge air, thereby providing a passive type valve function.

Moreover, the pumping means 11 may comprise two distinct components, one of which is configured to increase the pressure in the bladder 14, and the other one configured to release pressure. Alternatively, the pressurization and discharge components can be combined in a single pump 110.

### The Battery

As aforementioned, the device 1 according to the present invention may further comprise a power supply device 2 configured to supply power to the components of the wearable device 1 of the present invention.

Said power supply device 2 may comprise a built-in battery and/or a removable battery.

In one embodiment, the removable battery is rechargeable.

In one embodiment, the built-in battery has capacity comprised between 10 mAh and 300 mAh, preferably between 30 mAh and 100 mAh. This embodiment ensures that the built-in battery duration is long enough to ensure the continuity of the functions performed by the device 1 whilst the removable battery is not in place, for instance while the removable battery is being replaced or recharged. For instance, the continuity of the functions performed by the device 1 whilst the removable battery is not in place may be ensured for at least 30 minutes; preferably for at least 40 minutes, 50 minutes or 60 minutes.

The power supply device 2 may be buried inside the armband 10, partially exposed at the outer surface 14 or fully exposed at the outer surface 14. FIGS. 3A and 3B represent the device 1 according to the present invention in a particular embodiment in which the power supply device 2 is fully exposed at the outer surface, so that it can be removed without dismantling the armband 10. This is particularly advantageous to easily replace or recharge the battery.

### The Optical Sensor

As aforementioned, the device 1 comprises at least one optical sensor 12.

The optical sensor 12 advantageously allows to acquire a signal at an elevated acquisition frequency, which is suitable for a continuous or regular longitudinal monitoring of the wearer's blood pressure. Moreover, it does not cause discomfort to the wearer.

Blood pressure measurements based on the optical sensor signal may be provided as frequently as every 3 to 5 minutes if needed. In some cases, the blood pressure measurements may be provided at higher frequency, e.g., every minute or more often.

Preferably, the optical sensor 12 is a photoplethysmographic (PPG) sensor comprising a light source and a light detector, more preferably a single-wavelength PPG sensor.

For instance, the light source may be an infrared LED.

In one embodiment, the light source emits an infrared radiation from about 700 nm to about 1000 nm, preferably from about 900 nm to about 980 nm.

In one preferred embodiment, the light source is configured to emit an infrared radiation of about 940 nm.

The infrared radiation wavelengths herein mentioned preferably are the centroid wavelengths or the peak wavelengths of the light emitters at their operating temperature.

The device 1 may comprise several optical sensors 12, which may be structurally independent or embedded within a sensor array. In one particular example, the device 1 comprises at least three optical sensors and preferably: at least one infrared LED, at least one green LED, at least one red LED. Preferably, said infrared LED, green LED and red LED are configured to emit a radiation having a radiation wavelength of 940 nm, 536 nm, and 655 nm, respectively.

Advantageously, the optical sensor 12 may further comprise a light filtering element configured to minimize the intensity of the light reflected from the skin surface without affecting the light reflected by subcutaneous tissues.

The light filter element may comprise a first sheet of polarized glass placed on the light source of the optical sensor and/or a second sheet of polarized glass placed on the light detector of the optical sensor.

The first and second sheet of polarized glass have of opposing polarization so as to filter out the polarized light reflected by the skin surface without affecting the depolarized light backscattered from the deeper skin layers.

Moreover, the optical sensor 12 may further comprise a light filtering element placed on the light detector of the optical sensor 12 for filtering the wavelengths corresponding to a desired color component.

### The Pressure Sensor

As aforementioned, the present device 1 also comprises a pressure sensor 13. This allows to monitor the pressure in the bladder 14 and to obtain the blood pressure of the wearer therefrom, via an oscillometric technique (also called “cuff-based” technique).

Advantageously, the oscillometric technique allows to independently measure systolic and diastolic blood pressures. Moreover, the oscillometric technique provides an absolute blood pressure value.

The pressure sensor 13 may be of any type, for instance it may be a piezoresistive pressure sensor.

### Additional Sensors

The device 1 of the invention may further comprise at least one additional sensor 18 which is distinct from the at least one optical sensor 12 and the at least one pressure sensor 13 described above.

Some examples of said additional sensor 18 are provided in the following.

For instance, the device 1 may comprise a temperature sensor and/or a heat flux sensor. This embodiment is particularly advantageous to estimate a core body temperature of the wearer.

More precisely, in this embodiment, the processing unit 15 may further be configured to receive a signal from the temperature sensor and/or a signal from the heat flux sensor and to estimate a core body temperature of the wearer based on said received signals.

The device 1 may also comprises one or more additional sensors 18 including, without limitation: an ultrasound sensor; an accelerometer; a gyroscopic sensor; an electrical sensor, such as for instance an electrical sensor comprising electrodes; a tomographic sensor; an acoustic sensor; a magnetometer; a humidity sensor; an impedance sensor.

For instance, the device 1 may comprise a tomographic sensor or an acoustic sensor. In this particular example, the processing unit 15 may estimate an arterial noise based on the signal received from the tomographic sensor or the acoustic sensor, and it may characterize the blood flow based on said arterial noise.

Moreover, the processing unit 15 may receive sound data from the acoustic sensor, and estimate various physiological parameters of the wearer therefrom. For instance, the received sound data may be indicative of a turbulent blood flow or a laminar blood flow.

The acoustic sensor may be configured to record Korotkoff sounds during the deflation of the bladder 14.

In one embodiment, the device may comprise a sweat sensor. Said sweat sensor may be an impedance-based sensor comprising electrodes configured to emit and receive a small intensity current.

The device may further comprise one or more additional optical sensors distinct from the optical sensor 12 (e.g., they might have light sources configured to emit light radiations having different wavelengths).

In this case, the processing unit 15 may be configured to estimate various physiological parameters of the wearer, such as for example the blood oxygen saturation, based on the signals received from the additional optical sensors.

In one embodiment, the at least one optical sensor 12, the at least one pressure sensor 13 and the optional additional sensor 18 are integrated in a sensor array which is entirely 25 housed inside the armband 10, i.e., it is positioned within the inner surface 13 and the outer surface 14 of the armband 10. In this case, the inner surface 13 of the armband 10 comprises an opening 100 (FIG. 2C), configured to expose at least the optical sensor of the sensor array.

In some embodiments, the sensors 12, 13, 18 are not integrated in a single sensor array. For instance, the sensors 12, 13, 18 may be separated by a predefined distance along the armband length and/or the armband height, so that when the wearable device 1 is wrapped around an arm, said sensors 12, 13, 18 are distributed around said arm.

### The Processing Unit

As aforementioned, the present wearable device 1 further comprises a processing unit 15 that is configured to receive the signals from the sensors, estimating first and second blood pressure values therefrom, and triggering the inflation or deflation of the bladder 14 (for instance by opening or closing the valve 112 of the pumping means 11).

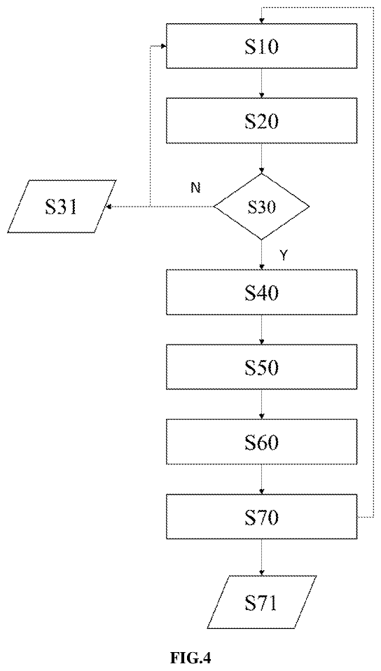

FIG. 4 illustrates the main operations which may be implemented by the processing unit 15 of the invention.

The processing unit 15 is configured to estimate physiological parameters of the wearer based on the signal received from the sensors 12, 13, 18. To this end, the processing unit 15 may be configured to execute two or more distinct algorithms

More in particular, the processing unit 15 is configured to receive S10 the signal from the optical sensor 12 and calculate S20 a first blood pressure value therefrom.

Preferably, the first blood pressure value is representative of one or more of: the systolic, diastolic and mean arterial pressure.

Said first blood pressure value is preferably calculated from a single optical sensor. In other words, the processing unit 15 is capable of calculating the first blood pressure value only from the signal from the optical sensor 12 (i.e., without using signals from other sensors).

The processing unit 15 may be configured to execute a machine learning algorithm in order to calculate the first blood pressure value.

Said machine-learning algorithm may be for instance a machine-learning model trained with the first blood pressure values previously obtained from a plurality of users as an input variable, and the corresponding second blood pressure values as an associated target output.

In addition to the blood pressure additional vital signs of the wearer may be estimated based on the signal received from the optical sensor and/or the signal from the pressure sensor 13, such as: the heart rate (HR), the respiratory rate (RR), the oxygen saturation (SpO2), and the like.

The processing unit 15 is further configured to monitor S30 the occurrence of events indicating that a calibration is required.

When one or more of said events is detected, the processing unit 15 may trigger S40 the inflation and the deflation of the bladder 14.

The processing unit 15 is further configured to receive S50 the signal from the pressure sensor 13 during the inflation and/or deflation of the bladder 14.

A second algorithm may be executed by the processing unit 15 to estimate S60 a second blood pressure value of the wearer based on the signal from the pressure sensor 13, i.e., via an oscillometric technique.

Since the second blood pressure value is obtained with an oscillometric technique, it is highly accurate and it is an absolute pressure value.

Moreover, the processing unit 15 is configured to calibrate S70 the first blood pressure value previously calculated based on said second blood pressure value.

In fact, the since the first blood pressure value is estimated based on the signal received from an optical sensor, a calibration is needed in order to obtain an accurate and absolute blood pressure value therefrom.

Advantageously, the present device 1 allows to automatically perform periodical, or repeated, calibrations of the first blood pressure value, at regular or variable time intervals.

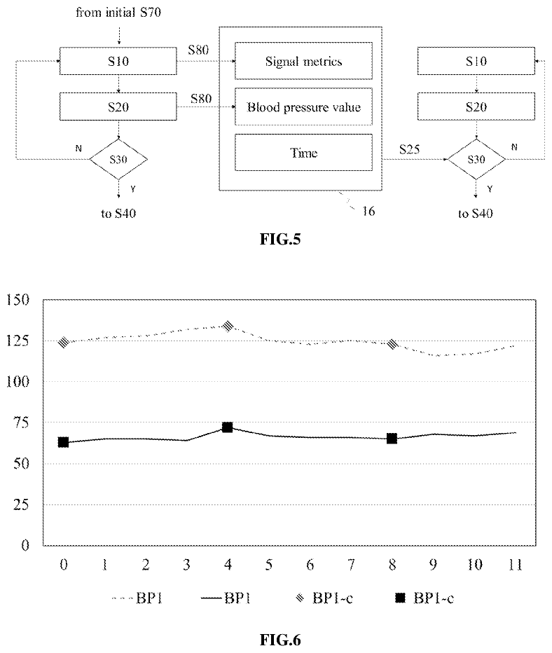

The calibration may be launched when one or more predefined criteria are met, for instance, such criteria may relate to:
(i) an interval of time received as input, for instance introduced as input by a healthcare provider, or calculated by a processing unit 15 (FIG. 6);
(ii) a signal metrics relating to the signal collected from a sensor, for instance the optical sensor or other sensors 18;
(iii) a physiological parameter of the wearer;
(iv) a temporal evolution of the signal metrics;
(v) a temporal evolution of the physiological parameter of the wearer; and/or
(vi) a temporal evolution of the perfusion index of the patient; which may be derived via a pulse oximeter and calculated as the ratio of a pulsatile blood flow to a non-pulsatile blood in peripheral tissue.

By “launching a calibration” it is meant performing the steps S40 to S70 described hereinabove.

Some of the exemplary criteria (i)-(vi) for launching a calibration will now be described in more details.

In a first example (i), the processing unit 15 is configured to trigger the inflation and deflation of the bladder 14 at the end of a predetermined time interval.

This time interval may be comprised between 0.25 hours and 8 hours.

In one embodiment, the predetermined time interval is comprised between 6 hours and 12 hours; preferably between 6 hours and 10 hours. This embodiment is advantageous when a lower calibration frequency is required, for instance when the wearer is a child.

Therefore, in this case the inflation and deflation of the bladder, the calibration of the first blood pressure value, are triggered at a fixed repetition frequency.

Moreover, in this embodiment, the inflation and deflation of the bladder 14 may be triggered independently of the characteristics of the signal received from the optical sensor 12.

With respect to the criterion (ii), in this case the processing unit 15 may be configured to:
calculate a value of the at least one metric related to the signal received from a first sensor, such for instance from the optical sensor 12;
compare the calculated value of the at least one metric with the respective threshold or range of values in the list of events;
based on the comparison, trigger the inflation and deflation of the bladder 14 by means of the pumping means 11.

In this embodiment, the processing unit 15 launches a calibration when a calculated signal metric is outside the respective range of values or threshold (i.e., of the range of values or threshold associated with said signal metric in the list of events).

Therefore, in this embodiment, the first blood pressure value may be calibrated at a variable calibration frequency.

The metric may be any statistical characteristic of a raw or filtered sensor signal, in the time domain or frequency domain.

For instance, the at least one metrics may be selected from a group comprising, but not limited to: maximum, minimum, slope, derivative, mean value, median value, coefficient of variation, signal-to-noise ratio, or a combination thereof, such as a linear or polynomial combination.

Moreover, the at least one signal metric may be a derivative, or a difference between a signal metric measured at the time interval t+Δt and a signal metric measured at a time t. Advantageously, this embodiment allows to launch a calibration when the received signal displays excessive variability over time.

The processing unit 15 may combine two or more of the aforementioned criteria.

For instance, it may modify the predetermined time interval of the first criterion (i) based on the metrics of the received signals as defined in criterion (ii).

Alternatively, the processing unit 15 may launch a calibration when the first criterion (i) is met, without modifying said time interval. In this case, if the signal received from one or more sensors 12, 18 meets the second criterion (ii) during the time interval, the processing unit 15 may trigger the inflation and deflation of the bladder 14, and restart the same time interval.

In regard to exemplary criterion (iii), the device 1 may comprise for instance a temperature sensor, and the processing unit 15 may increase or decrease the duration of the time interval based on the temperature of the wearer.

Furthermore, the device 1 may comprise one or more kinematic sensors such as an accelerometer. In this case, the processing unit 15 may be capable of detecting an activity level of the wearer based on the signal from the accelerometer, and it may increase or decrease the duration of the time interval based on said activity level.

This embodiment is particularly advantageous to adapt the frequency of calibration in response to a specific motion, exercise (e.g., climbing steps, cycling on an ergometer), postural change (e.g., from supine to seated or standing) or intervention (e.g., Valsalva maneuver, sustained handgrip).

In some examples, it is possible to manually launch calibration of the first blood pressure value (“on demand calibration”). This embodiment is advantageous since in some cases, the wearer or a healthcare specialist may need to launch the inflation and deflation of the bladder 14, without the occurrence of an event indicating that the calibration is required.

For instance, the wearer or a healthcare specialist may need to: register the absolute blood pressure and/or to estimate one or more parameters of the wearer therefrom.

The criteria for launching a calibration are preferably stored as a list of events in a processor-readable format. For instance, the list may comprise one or more signal metrics and a range of values associated with each signal metric.

In some cases, one or more criteria may comprise thresholds tagged as alert values (e.g., maximum variation in the first blood pressure value, or minimum intensity of the optical sensor signal). In this case, in addition to launching a calibration when one criterion is met, the processing unit 15 may also be configured to transmit an alert message. This embodiment advantageously allows to inform the wearer or the healthcare personnel that an action may be required (e.g., verifying the health status of the wearer if a sharp change in his/her first blood pressure value is detected, replacing a broken optical sensor 12).

As aforementioned, the processing unit 15 may trigger the inflation and deflation of the bladder 14 when a combination of events occurs.

The following example illustrates this embodiment. The processing unit 15 may be configured to monitor, i.e., measure, at least one signal metric during a first time interval and:
if, during the first time interval, the signal metric is outside the range of values associated with said signal metric in the list of events,
   prematurely end the first time interval;
   trigger the inflation and deflation of the bladder 14; and start a second time interval, the duration of the second time interval being retrieved from the list of events in the memory 16;
if, during the first time interval, the signal metric is not outside the range of values with said signal metric in the list of events,
   trigger the inflation and deflation of the bladder 14 at the end of the first time interval; and start a second time interval equal to the first time interval.

### Acquisition and Transmission Modules

The present device 1 may comprise an acquisition module configured to receive, as input, a request of calibration. For instance, the acquisition module may be embodied in an interface 17 of the device 1 (FIG. 1).

The acquisition module may also be configured to receive the wearer's data. The wearer's data may comprise, for instance: gender, age, weight.

The acquisition module may be also be configured to receive an electronic medical record of the wearer.

In this case, one or more algorithms executed by the processing unit 15 may be configured to estimate physiological parameters of the wearer based on the signal received from the sensors and on the received wearer's data.

In one embodiment, the acquisition module is further configured to receive data from a continuous or regular glucose monitoring device 1. Optionally, the power supply device 2 may be configured to supply power to the continuous or regular glucose monitoring device.

The present device 1 may also comprise a transmission module. The transmission module may be configured to transmit parameters estimated by the processing unit 15, such as for instance: the first blood pressure value and the second blood pressure value, or other parameters of the wearer. Preferably, the transmission module is a Bluetooth-based transmission module.

The transmission module may further be configured to transmit notifications to one or more recipient devices.

### The Set of Parameters

The present invention also allows to estimate a set of parameters of the wearer. Advantageously, the set of parameters of the wearer can be used to calibrate the first blood pressure value.

For instance, the set of parameters of the wearer may comprise parameters representative of hemodynamic property, mechanical property or geometrical property related to the arterial blood flow.

In this case, the estimated set of parameters may comprise: arterial compliance, arterial cross-sectional area, peripheral resistance, arterial stiffness, arterial wall thickness, blood flow, blood pressure, blood vessel elasticity index, cardiac output, elastic modulus of artery, heart rate, plasma volume, stroke volume, vessel length to radius ratio, and the like.

Furthermore, the estimated set of parameters of the wearer may comprise algorithmic parameters.

By “algorithmic parameter”, it is meant a measurable parameter derived by means of an algorithm which is related to, or summarizes, one or more physiological parameters.

In other words, algorithmic parameters do not necessarily represent a measure of a physiological parameter; however, they provide a numerical representation thereof.

For instance, algorithmic parameters may comprise feature parameters developed for machine learning purposes.

Moreover, the set of parameters may comprise a histogram statistic.

Examples of histogram statistics include, without limitation: 10^{th} percentile, 25^{th} percentile, 75^{th} percentile, 90^{th} percentile, entropy, intensity range, intensity variance, intensity standard deviation, kurtosis, mean absolute deviation, minimum intensity, maximum intensity, mean intensity, median intensity, root mean square, skewness, standard deviation, uniformity, variance.

### The Memory

The device 1 according to the present invention may further comprise a memory 16.

The memory 16 may be configured to store the list of events, or criteria, indicating that a calibration of the first blood pressure value is required.

Said list of events may comprise at least one time interval Δt and/or at least one signal metric and a range of values associated with said signal metric.

Furthermore, the memory may be configured to store one or more of the following:
an electronic medical record of the wearer;
hemodynamic properties, mechanical properties or geometrical properties;
algorithmic parameters;
histogram statistics;
person-specific parameters;
population-based parameters;
the most recent first blood pressure values of the wearer; for instance, at least the last 480, 240, 120, 60, or 30 first blood pressure values of the wearer.

### The Method

The present invention also relates to a method for estimation of a physiological parameter of a subject wearing the device 1 described hereinabove, notably for providing continuous or regular monitoring of his/her blood pressure.

More precisely, the method is for blood pressure estimation of a subject wearing a device 1 of the aforementioned type, the method being implemented by the processing unit 15 of said device 1.

FIG. 4 illustrates the main steps of the method according to a preferred embodiment.

The method comprises a step S10 of receiving, as input, a signal from a first sensor.

The method comprises a step S20 of calculating, by means of a first algorithm, a first blood pressure value of the wearer from the signal of the first sensor.

Preferably, said first sensor is an optical sensor 12.

In some embodiments, during step S10 a signal from at least one additional sensor 18 (e.g., a temperature sensor, an accelerometer, etc.) is also received. In this case, one or more physiological parameters of the wearer may be estimated based on the signal received from the first sensor.

The method further comprises a step S30 of monitoring the occurrence of an event indicating that a calibration is required.

The event indicating that a calibration is required may comprise a criterion related to the signal received from the first sensor. This embodiment allows to launch a cuff-based blood pressure measurement based on the signal collected from the first sensor, preferably an optic sensor. For instance, a calibration may be needed when a signal metric is abnormal, or when the temporal evolution over a predefined period of time is abnormal.

Based on the monitoring, the inflation and deflation of a bladder 14 is triggered S40.

During the inflation and/or the deflation of the bladder 14, a signal from a pressure sensor 13 is received S50.

During a second calculation step S60, a second blood pressure value of the wearer is calculated S60 based on the signal received from the pressure sensor 13.

Then, during step S70, the first blood pressure value is calibrated based on the second blood pressure value.

This calibration step S70 may also provide a set of parameters which can be used for calculating the future first blood pressure values (FIG. 5).

The calibration frequency may be constant or variable, depending on the occurrence of events indicating that a calibration is required.

The exemplary method of FIG. 4 also comprises outputting steps for outputting S31 the first blood pressure value and/or outputting S71 the second blood pressure value.

Of note, the calculation steps S20, S60 may be implemented by specific algorithms.

For instance, the step S20 of calculating the first blood pressure value is performed by a machine learning algorithm.

In this case, the first blood pressure value may be calculated based only on the signal from the optical sensor 12, via a step of decomposition into features and selection of applicable features. Advantageously, this embodiment allows to accurately estimate the blood pressure of the wearer while minimizing the number of required signals. In fact, the first blood pressure value is estimated based on a single optical sensor 12, thereby avoiding the use multiple optical sensors 12 placed at different location, which is expensive and may cause discomfort to the user.

In another embodiment, the step S20 of calculating a first blood pressure comprises:
calculating at least one blood pressure indicator based on the signal from the optical sensor 12,
estimating a first blood pressure of the wearer based of the wearer based on said at least one blood pressure indicator.

The blood pressure indicator may be calculated based on several signals. For instance, it may be calculated based on a first signal from the optical sensor 12 and a second signal from an ECG or another PPG sensor or an accelerometer. In this embodiment, the blood pressure indicator may be, for example: a pulse arrival time, a pulse transit time, a central pulse transit time, a peripheral pulse transit time.

The steps of the method may be repeated. After each repetition, an updated set of parameters of the wearer may be stored in the memory 16 and eventually replace a previously stored set of parameters of the wearer.

In this case, the first receiving step S10 and the first calculation step S20 are repeated at a first repetition frequency. The first repetition frequency is preferably comprised between 0.0005 s⁻¹ and 0.07 s⁻¹, more preferably between 0.001 s⁻¹ and 0.016 s⁻¹.

The monitoring step S30 may be performed at the same frequency of steps S10 and S20, or at a smaller frequency.

For instance, a selection of the signals received during step S10 and/or of the first blood pressure values calculated during step S20 may be applied beforehand, so that if e.g. the signal from the optical sensor 12 is acquired every second, one signal metric per minute may be exploited for the monitoring step S30.

FIG. 5 illustrates two consecutive implementations of the method of the invention, according to one particular embodiment.

In this example, the method also comprises a step S80 for storing a result of one of more than one steps S10 to S70 into the memory 16.

More precisely, a signal metric relating to the optical sensor signal, the first blood pressure value, and a time interval (e.g., the time at which the previous calibration step S70 was performed) are stored S80 in the memory.

Then, during a successive monitoring step S30, these results may be retrieved S25, and used as criteria to determine if a calibration should be performed.

In FIG. 5, the steps S40 to S70 are not illustrated for intelligibility purposes.

These steps (S40 to S70) are repeated at a second repetition frequency, the second repetition frequency being inferior to the first repetition frequency.

The second repetition frequency may be constant or variable.

For instance, if the event indicating that a calibration is required comprises a predetermined time interval, the second repetition frequency may be constant. If the event comprises a criterion relating to a sensor signal, the second repetition frequency may be variable.

In the present disclosure, the expressions “calibration frequency” and “second repetition frequency” have the same meaning.

In some examples, the method may also comprise the following steps (not illustrated):
receiving a signal from the temperature sensor and/or a signal from the heat flux sensor; and
estimating a core body temperature of the wearer at least based on said received signal.

Optionally, the estimation of the core body temperature of the wearer may take into account the signal received from the temperature sensor, the signal received from the heat flux sensor, and at least one vital sign of the wearer.

The estimation of the core body temperature of the wearer may be performed by a third algorithm distinct from the first and second algorithms In one embodiment, said third algorithm is a machine learning algorithm.

### Computer Program Product

The present invention also relates to a computer program product for performing the method as described above.

The computer program product may be written as computer programs, code segments, instructions or any combination thereof, for individually or collectively instructing or configuring a processor or computer to operate as a machine or special-purpose computer to perform the operations performed by hardware components.

In one example, the computer program product includes machine code that is directly executed by a processor or a computer, such as machine code produced by a compiler. In another example, the computer program product includes higher-level code that is executed by a processor or a computer using an interpreter. Programmers of ordinary skill in the art can readily write the instructions or software based on the block diagrams and the flow charts illustrated in the drawings and the corresponding descriptions in the specification, which disclose algorithms for performing the operations of the method as described above.

The present invention also relates to a computer-readable storage medium comprising instructions which, when the program is executed by a computer, cause the computer to carry out the steps of the method according to any one of the embodiments described hereabove.

The computer-readable storage medium is a non-transitory computer-readable storage medium.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium.

The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While various embodiments have been described and illustrated, the detailed description is not to be construed as being limited hereto. Various modifications can be made to the embodiments by those skilled in the art without departing from the true spirit and scope of the disclosure as defined by the claims.

### EXAMPLE

The present invention is further illustrated by the following case study.

One participant wearing the device of the present invention took part in the study. Signals were collected using a photoplethysmographic (PPG) optical sensor and a pressure sensor, both of which were embedded within the armband.

FIG. 6 shows the first blood pressure value (in mm Hg) of the participant versus time (in hours) obtained during an observation period of 11 hours, and more precisely:
the diastolic blood pressure value calculated based on the signal from the optical sensor 12 (solid line, reference: BP1);
the systolic blood pressure value calculated based on the signal from the optical sensor 12 (dotted line, reference: BP1); p1 the calibrated diastolic blood pressure value (black squares, reference BP1-c); and
the calibrated systolic blood pressure value (grey diamonds, reference BP1-c).

In this example, cuff-based measurements were triggered every 4 hours, and the first blood pressure values (systolic and diastolic) calibrated therefrom.

Moreover, each of these calibration steps outputs a set of parameters which are used to calculate, along with the signal from the optical sensor 12, the first blood pressure values between two consecutive calibrations.

These results show that the invention provides a continuous and accurate blood pressure monitoring with a single device (e.g., without sensors at multiple locations on the subject's body, and without external calibration standards).

1-16. (canceled)

17. A wearable device for blood pressure measurement comprising:
an armband with an inner surface and an outer surface;
a bladder within the armband configured to be inflated and deflated;
pumping means for inflating and deflating said bladder, the pumping means comprising a pump, a pipe in fluid communication with the bladder and a valve;
at least one optical sensor within the armband, the optical sensor being at least partially exposed at the inner surface of the armband, so as to permit acquisition of an optic signal from an arm of the subject wearing the armband;
at least one pressure sensor configured to acquire a signal indicative of a pressure within the bladder; and
a processing unit configured to:
receive the signal from the optical sensor and the signal from the pressure sensor;
calculate a first blood pressure value of the wearer based on the signal from the optical sensor, and a second blood pressure value based on the signal from the pressure sensor; and
calibrate the first blood pressure value on the basis of the second blood pressure value.

18. The device according to claim 17, wherein the pumping means are within the armband.

19. The device according to claim 17, further comprising a power supply device, the power supply device preferably comprising a built-in battery and a removable battery.

20. The device according to claim 17, further comprising a memory configured to store a list of events, the list of events comprising at least one time interval Δt, wherein processing unit is configured to:
retrieve, from the memory, the list of events and monitor the occurrence of said events;
in response to the detection of at least one of said events, trigger the inflation and deflation of the bladder by means of the pumping means;
receive the signal from the pressure sensor during said inflation and/or deflation of the bladder;
calculate the second blood pressure value of the wearer based on the signal from the pressure sensor.

21. The device according to claim 20, wherein processing unit is further configured to:
execute a first algorithm configured to calculate the first blood pressure value of the wearer based on the signal received from the optical sensor;
execute a second algorithm configured to calculate the second blood pressure value of the wearer based on the signal received from the pressure sensor;
estimate a set of parameters of the wearer based on the first and the second blood pressure values.

22. The device according to claim 21, wherein the processing unit is configured to calibrate the first blood pressure value based on the estimated set of parameters of the wearer.

23. The device according to claim 17, wherein the device further comprises a temperature sensor and/or a heat flux sensor; and wherein the processing unit is further configured to receive a signal from the temperature sensor and/or a signal from the heat flux sensor and to execute a third algorithm to estimate a core body temperature of the wearer based on said received signal.

24. The device according to claim 17, wherein the device further comprises at least one third sensor selected from a group comprising: an ultrasound sensor, a accelerometer, a gyroscopic sensor, an electrical sensor, an optical sensor, a tomographic sensor, an acoustic sensor, a magnetometer, a humidity sensor; and wherein the processing unit is further configured to receive a signal from said third sensor and to estimate a physiological parameter based on said received signal.

25. A method for continuous or regular monitoring of the blood pressure of a subject, the method being implemented by a wearable device according to claim 17 and comprising the following steps:
a) receiving, as input, a signal from the optical sensor;
b) calculating the first blood pressure value of the wearer by means of a first algorithm based on the signal received from the optical sensor;
c) receiving, as input, a signal from the pressure sensor during deflation and/or inflation of the bladder;
d) calculating the second blood pressure value of the wearer by means of a second algorithm based on the signal received from the pressure sensor; and
e) calibrating the first blood pressure value on the basis of the second blood pressure value.

26. The method according to claim 25, further comprising the following steps:
retrieve a list of events stored in a memory, wherein in the list of events each event is associated to at least one predefined threshold, and monitoring the occurrence of said events; and
triggering the inflation and deflation of the bladder if the signal from or the first blood pressure value is above or below the predefined threshold.

27. The method according to claim 25, further comprising a step of receiving, as input, a signal from a third sensor, said third sensor being distinct from the optical sensor and the pressure sensor, and wherein during the calculation step of the first blood pressure value, the first blood pressure value of the wearer is calculated by means of a first algorithm based on the signal received from the optical sensor and/or on the signal received from the third sensor.

28. The method according to claim 25, wherein the steps of the methods are preceded by a step of retrieving a set of parameters of the wearer from a memory; and
wherein during the calculation step of the first blood pressure value, the first blood pressure value of the wearer is calculated based on the signal received from the optical sensor and on the retrieved set of parameters.

29. The method according to claim 25, wherein in the step of calculating the first blood pressure value and the step of calculating the second blood pressure value, the first and the second blood pressure values are performed by means of a machine learning algorithm.

30. The method according to claim 26, wherein the receiving step and the calculating step first blood pressure value are repeated at a first repetition frequency and the step of monitoring to the step of calibrating are repeated at a second repetition frequency, the second repetition frequency being inferior to the first repetition frequency.

31. A computer-readable storage medium comprising instructions which, when the program is executed by a computer, cause the computer to carry out the steps of the method according to claim 25.
